(19) United States
(12) Patent Application Publication
(10) Pub. No.: US 2010/0035888 A1
(43) Pub. Date: Feb. 11, 2010

(54) Diaryl Urea for Treating Pulmonary Hypertension

(75) Inventors: Peter Sandner, Wuppertal (DE); Hanna Tinel, Wuppertal (DE); Joachim Hütter, Wuppertal (DE); Bernd Riedl, Wuppertal (DE); Martina Klein, Dusseldorf (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bater Healthcare AG, Leverkusen (DE)

(21) Appl. No.: 12/084,662

(22) PCT Filed: Oct. 30, 2006
(86) PCT No.: PCT/EP2006/010406

§ 371 Date: Mar. 16, 2009

(30) Foreign Application Priority Data
Nov. 10, 2005 (EP) 05024509.1
Dec. 15, 2005 (EP) 05027450.5
Jun. 14, 2006 (EP) 06012234.8

(51) Int. Cl. A61K 31/4412; A61K 31/496; A61K 31/519; A61K 31/506; A61P 7/00

(52) U.S. Cl. 514/251; 514/350; 514/252.16; 514/269

(57) ABSTRACT

The present invention relates to pharmaceutical compositions for treating, preventing or managing pulmonary hypertension comprising 4{4-[3-(4-chloro-3-trifluoromethylphenyl)-ureido]-3-fluorophenoxy}-pyridine-2-carboxylic acid methylamide optionally combined with at least one additional therapeutic agent.

The present invention relates to pharmaceutical compositions and combinations for treating, preventing or managing pulmonary hypertension comprising 4{4-[3-(4-chloro-3-trifluoromethylphenyl)ureido]-3-fluorophenoxy}-pyridine-2-carboxylic acid methylamide optionally combined with at least one additional therapeutic agent.

Diaryl urea compounds e.g. 4{4-[3-(4-chloro-3-trifluoromethylphenyl)-ureido]-3-fluorophenoxy}-pyridine-2-carboxylic acid methylamide as described e.g. in US 20050038080 are potent anti-cancer and anti-angiogenic agents that possess various activities, including inhibitory activity on the VEGFR, PDGFR, raf, p38, and/or flt-3 kinase signaling molecules. These diaryl urea compounds have been previously characterized as having various activities, including for inhibiting the Raf/MEK/ERK pathway, raf kinase, p38 kinase, VEGFR kinase, PDGFR kinase. These activities and their use in treating various diseases and conditions are disclosed in, e.g., WO 2005/009961.

Pulmonary hypertension refers to a disease characterized by sustained elevations of pulmonary artery pressure (L. J. Rubin, The New England Journal of Medicine, 1997, 336(2), 111). Current treatment of pulmonary hypertension depends on the stage and the mechanism of the disease. Typical treatments for pulmonary hypertension include anticoagulation, oxygen supplementation, conventional vasodilator therapy, transplantation and surgical care. Therapeutic agents presently used for the treatment of pulmonary hypertension include e.g. calcium channel blockers and pulmonary vasodilators

The present invention provides pharmaceutical compositions for treating, preventing or managing pulmonary hypertension comprising a compound of formula I and optionally at least one further therapeutic agent.

The present invention can be used e.g. by administering a diaryl urea compound of formula I and optionally a further therapeutic agent, pharmaceutically-acceptable salts thereof, and derivatives thereof, etc.

The compounds with the structure of formula I, pharmaceutically acceptable salts, polymorphs, solvates, hydrates metabolites and prodrugs thereof, including diastereoisomeric forms (both isolated stereoisomers and mixtures of stereoisomers) are collectively referred to herein as the “compounds of formula I”.

Formula (I) is as follows:

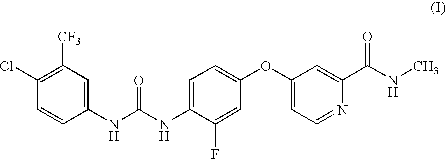

Where the plural form of the word compounds, salts, and the like, is used herein, this is taken to mean also a single compound, salt, or the like.

The present invention also relates to useful forms of the compounds as disclosed herein, such as pharmaceutically acceptable salts, metabolites and prodrugs. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoromethanesulfonate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aryl or aralkyl halides like benzyl and phenethyl bromides and others monosubstituted aralkyl halides or polysubstituted aralkyl halides.

Solvates for the purposes of the invention are those forms of the compounds where solvent molecules form a complex in the solid state and include, but are not limited to for example ethanol and methanol. Hydrates are a specific form of solvates, where the solvent molecule is water.

Certain pharmacologically active agents can be further modified with labile functional groups that are cleaved after in vivo administration to furnish the parent active agent and the pharmacologically inactive derivatizing group. These derivatives, commonly referred to as prodrugs, can be used, for example, to alter the physicochemical properties of the active agent, to target the active agent to a specific tissue, to alter the pharmacokinetic and pharmacodynamic properties of the active agent, and to reduce undesirable side effects. Prodrugs of the invention include, e.g., the esters of appropriate compounds of this invention that are well-tolerated, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

Methods which can be used to synthesize other prodrugs are described in the following reviews on the subject, which are incorporated herein by reference for their description of these synthesis methods:
Higuchi, T.; Stella, V. eds. Prodrugs As Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: N.Y. (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff, M. E. ed. Burgers Medicinal Chemistry And Drug Discovery John Wiley & Sons: New York (1997), 949-982.

The metabolites of the compounds of this invention include oxidized derivatives of the compounds of formula I, wherein one or more of the nitrogens are substituted with a hydroxy group; which includes derivatives where the nitrogen atom of the pyridine group is in the oxide form, referred to in the art as 1-oxo-pyridine or has a hydroxy substituent, referred to in the art as 1-hydroxypyridine.

### General Preparative Methods

The compounds of the invention may be prepared by use of known chemical reactions and procedures as described e.g. in the following published international application WO 2005/009961.

### Further Therapeutic Agents

The compounds of formula I according to the present invention can be combined with further therapeutic agents presently used to treat, prevent or manage pulmonary hypertension such as, but not limited to, anticoagulants, diuretics, cardiac glycosides, calcium channel blockers, vasodilators, prostacyclin analogues, endothelium antagonists, phosphodiesterase inhibitors, endopeptidase inhibitors, lipid lowering agents, thromboxane inhibitors and other therapeutics known to reduce pulmonary artery pressure.

Examples of anticoagulants include, but are not limited to, e.g. warfarin useful in the treatment of patients with pulmonary hypertension having an increased risk of thrombosis and thromboembolism.

Examples of calcium channel blockers include, but are not limited to, diltiazem, felodipine, amlodipine and nifedipine particularly useful for vasoreactive patients at right heart catheterization.

Examples of vasodilators include, but are not limited to, e.g. prostacyclin, epoprostenol, treprostinil and nitric oxide (NO).

Examples of phosphodiesterase inhibitors include, but are not limited to, particularly phosphodiesterase V inhibitors such as e.g. tadalafil, sildenafil and vardenafil.

Examples of endothelin antagonists include, but are not limited to, e.g. bosentan and sitaxentan, preferably bosetan.

Examples of prostacyclin analogues include, but are not limited to, e.g. ilomedin, treprostinil and epoprostenol.

Examples of lipid lowering agents include, but are not limited to, e.g. HMG CoA reductase inhibitors such as simvastatin, pravastatin, atorvastatin, lovastatin, itavastatin, fluvastatin, pitavastatin, rosuvastatin, ZD-4522 and cerivastatin

Examples diuretics include, but are not limited to, e.g. chlorthalidon, indapamid, bendroflumethiazid, metolazon, cyclopenthiazid, polythiazid, mefrusid, ximapid, chlorothiazid and hydrochlorothiazid particularly useful to manage peripheral edema.

Examples of other therapeutics known to reduce pulmonary artery pressure include, but are not limited to, e.g. ACE inhibitors such as enalapril, ramipril, captopril, cilazapril, trandolapril, fosinopril, quinapril, moexipril, lisinopril and perindopril, or AT II inhibitors such as losartan, candesartan, irbesartan, embusartan, valsartan and telmisartan, or iloprost, betaprost, L-arginine, omapatrilat, oxygen particularly useful in those patients with resting or exercise-induced hypoxemia or digoxin particularly useful to improve right ventricular function in patients with right ventricular failure.

Furthermore the compounds and combinations of the invention can be combined with kinase inhibitors and/or elastase inhibitors.

Examples of kinase inhibitors include, but are not limited to, e.g. BMS-354825, canertinib, erlotinib, gefitinib, imatinib, lapatinib, lestaurtinib, lonafarnib, pegaptanib, pelitinib, semaxanib, tandutinib, tipifarnib, vatalanib, lonidamine, fasudil, leflunomide, bortezomib, imatinib, erlotinib and glivec. Preference is given to glivec.

### Indications

The compounds and combinations according to the present invention can be used for manufacture of a medicament for treating, preventing and managing pulmonary hypertension. Also the present invention provides methods of treating, preventing and managing pulmonary hypertension, comprising administering effective amounts of at least one compound of formula I and optionally at least one further therapeutic agent according to the invention. An “effective amount” is the quantity of the compound that is useful to achieve the desired result, e.g., to treat, prevent or manage the disease or condition.

The term “pulmonary hypertension” according to the invention include, but is not limited to, primary pulmonary hypertension, secondary pulmonary hypertension, familial pulmonary hypertension, sporadic pulmonary hypertension, precapillary pulmonary hypertension, pulmonary arterial, pulmonary artery hypertension, idiopathic pulmonary hypertension, thrombotic pulmonary-arteriopathy, plexogenic pulmonary arteriopathy and pulmonary hypertension associated with or related to, left ventricular dysfunction, mitral valvilar disease, constrictivepericarditis, aortic stenosis, cardiomyopathy, mediastinal fibrosis, anomalous pulmonary venous drainage, pulmonary venoocclusive disease, collagen vascular disease, congenital heart disease, congenital heart disease, pulmonary venus hypertension, chronic obstructive pulmonary disease, interstitial lung disease, sleep-disordered breathing, alveolarhyperventilation disorder, chronic exposure to high altitude, neonatal lung disease, alveolar-capillary dysplasia, sickle cell disease, other coagulation disorders, chronic thromboemboli, connective tissue disease, lupus, schistosomiasis, sarcoidosis or pulmonary capillary hemangiomatosis.

Any form of pulmonary hypertension can be treated in accordance with the present invention, including, but not limited to, mild, e.g., associated with increases of mean blood pressure of about 20-30 mm Hg at rest; moderate, e.g., associated with increases of 30-39 mm Hg at rest; and severe, e.g., associated with increases of 40 mm Hg or more at rest.

Pulmonary hypertension includes pulmonary arterial hypertension (PAH), and includes, primary pulmonary hypertension (PPH), idiopathic PAH (IPAH), familial PAH (FPAH). Several classifications systems for pulmonary hypertension have been published, including the Evian Nomenclature and Classification of pulmonary hypertension (PH) (1998) and the Revised Nomenclature and Classification of PH (2003). See, Lewis et al., Chest, 2004, 126, 73-10, which is hereby incorporated by reference in its entirety. Any disease PH listed in these classification schemes can be treated, managed, or prevented in accordance with the present invention. Risk factors and diagnostic criteria for PH are described in McGoon et al., Chest, 126, 14-34, 2004, which is hereby incorporated by reference in its entirety.

The following list is the 2003 classification proposed at the Third World Conference on Pulmonary Hypertension: PAH, IPAH, FPAH, collagen vascular disease, congenital systemic to pulmonary shunts (large, small, repaired or nonrepaired), Portal hypertension, drugs and toxins, other (glycogen storage disease, gaucher disease, hereditary hemorrhagic telangiectasia, hemoglobinopathies, myeloproliferative disorders, splenectomy), associated with significant venous or capillary involvement, pulmonary venous hypertension, pulmonary capillary hemangiomatosis, pulmonary venous hypertension, left-sided atrial ventricular heart disease, left-sided valvular heart disease, pulmonary hypertension associated with hypoxemia, COPD, interstitial lung disease, sleep-disordered breathing, alveolar hypoventilation disorders, chronic exposure to high altitude, PH due to chronic thrombotic and/or embolic disease, thromboembolic obstruction of proximal pulmonary arteries, thromboembolic obstruction of distal pulmonary arteries, pulmonary embolism (tumor, parasites, foreign material), sarcoidosis, histiocytosis X, lymphangiomatosis, compression of pulmonary vessels (adenopathy, tumor, fibrosing mediastinitis)

Any of the above-mentioned disorders can be associated with an increased risk of pulmonary hypertension, including, subjects having, e.g., congenital heart disease (e.g., Eisenmenger syndrome); left heart disease; pulmonary venous disease (e.g., fibrosis tissue narrowing or occluding pulmonary veins and venules); pulmonary arterial disease; diseases causing alveolar hypoxia; fibrotic lung diseases; Williams syndrome; subjects with intravenous drug abuse injury; pulmonary vasculitis (such as Wegener's, Goodpasture's, and Churg-Strauss syndromes); emphysema; chronic bronchitis; kyphoscoliosis; cystic fibrosis; obesity-hyper-ventilation and sleep apnea disorders; pulmonary fibrosis; sarcoidosis; silocosis; CREST (calcinosis cutis, Raynaud phenomenon; esophageal motility disorder; sclerodactyly, and teleangiectasia) and other connective tissue diseases. For example, a subject who possesses a BMPR2 mutation (bone morphogenetic protein receptor II) has a 10-20% lifetime risk of acquiring FPAH. Subjects with hereditary hemorrhagic telangiectasa were also identified as being at risk for IPAH, especially those carrying mutations in ALK1. See, McGoon et al., Chest, 2004, 126, 14-34.

According to the invention the term “treating” refers to the administration of a pharmaceutical composition after the onset of symptoms of pulmonary hypertension, whereas “preventing” refers to the administration prior to the onset of symptoms, particularly to patients at risk of pulmonary hypertension. The term “managing” encompasses preventing the recurrence of pulmonary hypertension in a patient who suffered from pulmonary hypertension.

### Administration

Compounds or drug combinations of the present invention can be administered in any form by any effective route, including, e.g., oral, parenteral, enteral, intravenous, intraperitoneal, topical, transdermal (e.g., using any standard patch), ophthalmic, nasally, local, non-oral, such as aerosol, inhalation, subcutaneous, intramuscular, buccal, sublingual, rectal, vaginal, intra-arterial, and intrathecal, etc. They can be administered alone, or in combination with any ingredient(s), active or inactive.

Preference is given to an oral administration.

Compounds or drug combinations of the present invention can be converted in a known manner into the usual formulations, which may be liquid or solid formulations e.g. without limitation normal and enteric coated tablets, capsules, pills, powders, granules, elixirs, tinctures, solution, suspensions, syrups, solid and liquid aerosols and emulsions.

Examples of solid formulations for oral administration are described in U.S. provisional application No. 60/605,752.

The combinations of the present invention can be administered at any time and in any effective form. For example, the compounds can be administered simultaneously, e.g., as a single composition or dosage unit (e.g., a pill or liquid containing both compositions), or they can be administered as separate compositions, but at the same time (e.g., where one drug is administered intravenously and the other is administered orally or intramuscularly). The drugs can also be administered sequentially at different times. Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The drug combinations can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy.

Compounds or drug combinations of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

In addition, compounds or drug combinations of the present invention can be administered with other active agents or other therapies that are utilized to treat any of the above-mentioned diseases and/or conditions.

Other therapies according to the invention include, but are not limited to, e.g. physical or mechanical therapy such as electrical stimulation, acupuncture, magnet therapy or topical use of polyurethane films.

The present invention provides also combinations of at least one compound of Formula I and at least one other therapeutic agent mentioned above useful in treating a disease or disorder. “Combinations” for the purposes of the invention include:
single compositions or dosage forms which contain at least one compound of Formula I and at least one other therapeutic agent mentioned above;
combination packs containing at least one compound of Formula I and at least one other therapeutic agent mentioned above to be administered concurrently or sequentially;
kits which comprise at least one compound of Formula I and at least one other therapeutic agent mentioned above packaged separate from one another as unit dosages or as independent unit dosages, with or without instructions that they be administered concurrently or sequentially; and
separate independent dosage forms of at least one compound of Formula I and at least one other therapeutic agent mentioned above which cooperate to achieve a therapeutic effect, e.g., treatment of the same disease, when administered concurrently or sequentially.

The dosage of each agent of the combination can be selected with reference to the other and/or the type of disease and/or the disease status in order to provide the desired therapeutic activity. For example, the active agents in the combination can be present and administered in a fixed combination. “Fixed combination” is intended here to mean pharmaceutical forms in which the components are present in a fixed ratio that provides the desired efficacy. These amounts can be determined routinely for a particular patient, where various parameters are utilized to select the appropriate dosage (e.g., type of disease, age of patient, disease status, patient health, weight, etc.), or the amounts can be relatively standard.

The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

Preference is given to an amount of the compound of formula I from 20 to 2000 mg, preferably from 40 to 800 mg, more preferably from 50 to 600 mg.

Particular preference is given to an amount of 4{4-[3-(4-chloro-3-trifluoromethylphenyl)-ureido]-3-fluorophenoxy}-pyridine-2-carboxylic acid methylamide in the pharmaceutical composition from 20 to 3000 mg, preferably from 50 to 1500, more preferably from 60 to 1000 mg.

In another embodiment of the invention the compound of formula I is administered in combination with at least one further therapeutic agent in an amount that those of ordinary skill in the art can determine by their professional judgement.

The pharmaceutical composition according to the invention is administered one or more, preferably up to three, more preferably up to two times per day. Preference is given to an administration via the oral route. With each administration the number of tablets or capsules taken in at the same time should not exceed two.

Nevertheless, it may in some cases be advantageous to deviate from the amounts specified, depending on body weight, individual behaviour toward the active ingredient, type of preparation and time or interval over which the administration is effected. For instance, less than the aforementioned minimum amounts may be sufficient in some cases, while the upper limit specified has to be exceeded in other cases. In the case of administration of relatively large amounts, it may be advisable to divide these into several individual doses over the day.

The combination can comprise effective amounts of at least one compound of Formula I and at least one other therapeutic agent mentioned above, which achieves a greater therapeutic efficacy than when either compound is used alone. The combination can be useful to treat, prevent or manage pulmonary hypertension, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating, preventing or managing pulmonary hypertension where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising a compound of formula I and at least one compound selected from the group consisting of phosphodiesterase V inhibitors, endothelin antagonists, prostacyclin analogues, kinase inhibitors and elastase inhibitors. More preferably a combination comprising 4{4-[3-(4-chloro-3-trifluoromethylphenyl)-ureido]-3-fluorophenoxy}-pyridine-2-carboxylic acid methylamide and at least one compound selected from the group consisting of tadalafil, sildenafil, vardenafil, bosentan, sitaxentan, ilomedin, treprostinil and epoprostenol is used. Most preferably a combination comprising 4{4-[3-(4-chloro-3-trifluoromethylphenyl)-ureido]-3-fluorophenoxy}-pyridine-2-carboxylic acid methylamide and bosentan or vardenafil is used.

### EXAMPLES

The effects of the compounds and drug combinations according to the invention are tested in vitro on isolated rat pulmonary arteries and in vivo in monocrotaline-treated rats with pulmonary hypertension.

### Isolated Small Pulmonary Arteries

Male Wistar rats (250-300 g) are anaesthetized with ether, and the lungs are removed. The left pulmonary arterial vessel is dissected and placed in ice-cold Krebs-Henseleit (KH) buffer of following composition (in mmol/l): NaCl 112, KCl 5.9, CaCl₂ 2.0 MgCl₂ 1.2, NaH₂PO₄ 1.2, NaHCO₃ 25, glucose 11.5 and optionally the compound/combination to be tested in a concentration of 10⁻¹⁰ to 10⁻⁴ mol/l.

For measurement of isometric tension, ring segments, 2 mm in length, are mounted in a small vessel chamber myograph. Two wires (40 μm diameter) are introduced through the lumen of the segments and mounted according to the method described by Mulvany and Halpern (Circulation Research 1977; 41:19-26). After a 30 min equilibration period in oxygenated KH solution at 37° C. and pH=7.4, segments are stretched to their optimal lumen diameter for active tension development which is determined based on the internal circumference-wall tension ratio of the segments by setting their internal circumference to 90% of what the vessels would have if they are exposed to a passive tension equivalent to that produced by a transmural pressure of 30 mmHg.

Afterwards, segments are washed three times with KH solution and left to equilibrate for 30 min. Segment contractility is then tested by an initial exposure to a high K⁺ solution (120 mmol/l K⁺-KH solution, which is identical to KH solution except that NaCl is replaced by KCl on an equimolar basis).

The vessels are than pre-contracted using K⁺ (50 mmol/l) KH solution. When the contraction is stabilized, an accumulative dose response curve of the compound/combination tested is constructed. The stabilized contraction induced by K⁺ (50 mmol/l) KH solution is defined as 100% tension. The relaxation is expressed as percentage tension.

### Pulmonary Artery Pressure in Monocrotaline Treated Rats

Male Sprague Dawley rats (250-300 g) are treated with monocrotaline 60 mg/kg subcutanously (=day 0). On day 14 after monocrotaline injection treatment the compound/combination to be tested is administered. On day 28 hemodynamic parameters, i.e. right ventricular pressure, systemic blood pressure, heart rate, arterial and venous oxygen saturation are measured and compared with untreated control animals.

### Results:

Monocrotaline (MCT) treated rats are randomized to receive 4{4-[3-(4-chloro-3-trifluoromethylphenyl)-ureido]-3-fluorophenoxy}-pyridine-2-carboxylic acid methylamide 3 mg/kg or vehicle by gavage once daily after the onset of moderate pulmonary arterial hypertension starting 14 days after the injection of MCT until day 28. In animals with MCT-induced pulmonary arterial hypertension treatment with 4{4-[3-(4-chloro-3-trifluoromethylphenyl)-ureido]-3-fluorophenoxy}-pyridine-2-carboxylic acid methylamide markedly decreases right ventricular hypertrophy, compared to vehicle treated animals (right ventricle/left ventricle+septum ratio control: 0.25±0.01; 4{4-[3-(4-chloro-3-trifluoromethylphenyl)-ureido]-3-fluorophenoxy}-pyridine-2-carboxylic acid methylamide: 0.28±0.01 vs. placebo: 0.62±0.02) (mean±SEM). This effect of 4{4-[3-(4-chloro-3-trifluoromethylphenyl)-ureido]-3-fluorophenoxy}-pyridine-2-carboxylic acid methylamide is paralleled by an improvement of the survival of the animals (mortality rate control: 0%; BAY73-4506: 0% vs. Placebo: 40%).

### Example 1

### Preparation of a 4:1 co-precipitate formulation solid dispersion of 4{4-[3-(4-chloro-3-trifluoromethylphenyl)-ureido]-3-fluorophenoxy}-pyridine-2-carboxylic acid methyl amide with polyvinylpyrrolidone

In an uncapped vial, one part of 4{4-[3-(4-chloro-3-trifluoromethylphenyl)-ureido]-3-fluorophenoxy}-pyridine-2-carboxylic acid methyl amide as a free base was mixed with four parts polyvinylpyrrolidone (PVP-25/Kollidon 25), and dissolved in a sufficient amount of a 1:1 mixture of acetone and ethanol, until all powders are in solution. The uncapped vial was placed into a vacuum oven set at 40° C., and let dry for at least 24-48 hours.

1. A pharmaceutical composition for treating, preventing or managing pulmonary hypertension comprising a compound of formula I or a pharmaceutically acceptable salt, polymorph, solvate, hydrate, metabolite, prodrug or diastereoisomeric form thereof and an elastase inhibitor and/or a kinase inhibitor,
wherein said compound of formula I is:

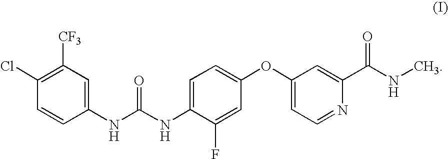

2. (canceled)

3. The pharmaceutical composition of claim 1 wherein the kinase inhibitor is glivec.

4. The pharmaceutical composition of claim 1 further comprising at least one therapeutic agent selected from the group consisting of anticoagulants, diuretics, cardiac glycosides, calcium channel blockers, vasodilators, prostacyclin analogues, endothelium antagonists, phosphodiesterase inhibitors, endopeptidase inhibitors, lipid lowering agents, thromboxane inhibitors and a therapeutic known to reduce pulmonary artery pressure.

5. The pharmaceutical composition of claim 4 wherein the therapeutic agent is a phosphodiesterase V inhibitor, endothelin antagonist or prostacyclin analogue.

6. The pharmaceutical composition of claim 4 wherein the therapeutic agent is tadalafil, sildenafil, vardenafil, bosentan, sitaxentan, ilomedin, treprostinil or epoprostenol.

7. (canceled)

8. (canceled)

9. (canceled)

10. A method for treating, preventing or managing pulmonary hypertension in a subject in need thereof comprising administering effective amounts of a compound of formula I or a pharmaceutically acceptable salt, polymorph, solvate, hydrate, metabolite, prodrug or diastereoisomeric form thereof and an elastase inhibitor and/or a kinase inhibitor,
wherein said compound of formula I is:

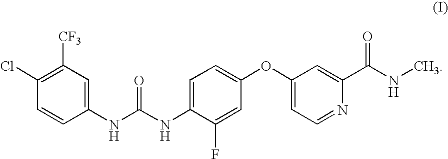

11. (canceled)

12. The method of claim 10 wherein the compound of formula I is additionally combined with at least one therapeutic agent selected from the group consisting of anticoagulants, diuretics, cardiac glycosides, calcium channel blockers, vasodilators, prostacyclin analogues, endothelium antagonists, phosphodiesterase inhibitors, endopeptidase inhibitors, lipid lowering agents, thromboxane inhibitors and a therapeutic known to reduce pulmonary artery pressure.

13. The method of claim 10, wherein the kinase inhibitor is glivec.

14. The method of claim 12, wherein the therapeutic agent is a phosphodiesterase V inhibitor, endothelin antagonist or prostacyclin analogue.

15. The method of claim 12, wherein the therapeutic agent is tadalafil, sildenafil, vardenafil, bosentan, sitaxentan, ilomedin, treprostinil or epoprostenol.
